(19) United States
(12) Patent Application Publication
(10) Pub. No.: US 2007/0260144 A1
(43) Pub. Date: Nov. 8, 2007

(54) Acoustic Control of Emboli in Vivo

(75) Inventors: Nathan Sela, Modiin (IL); Michael Kardosh, Kiryat Ono (IL); Simcha Milo, Haifa (IL)

Correspondence Address: DARBY & DARBY P.C., P.O. BOX 770, Church Street Station, New York, NY, 10008-0770, US

(73) Assignee: NEUROSONIX LTD., ARIEL (IL)

(21) Appl. No.: 10/597,801

(22) PCT Filed: Feb. 9, 2005
(86) PCT No.: PCT/IL05/00163

§ 371 Date: Jun. 1, 2007

Related U.S. Application Data
(60) Provisional application No. 60/544,459, filed on Feb. 12, 2004.
(60) Provisional application No. 60/572,283, filed on May 17, 2004.

(51) Int. Cl. A61B 8/14

(52) U.S. Cl. 600/472

(57) ABSTRACT

A device (30) for controlling a flow of emboli (48) in an aorta (36) of a patient. The device includes an ultrasonic transducer (44), which is configured to transmit an ultrasonic beam (52) into the aorta in a vicinity of a great origin of a neck vessel (38). A driver circuit (58) is coupled to drive the ultrasonic transducer to generate the ultrasonic beam at a frequency and power level sufficient to divert at least a target fraction of the emboli of a given type and size away from the neck vessel.

### CROSS-REFERENCE TO RELATE APPLICATIONS

This application claims the benefit of U.S. Provisional Patent Application 60/544,459, filed Feb. 12, 2004, and of U.S. Provisional Patent Application 60/572,283, filed May 17, 2004. This application is a continuation-in-part of U.S. patent application Ser. No. 10/162,824, filed Jun. 4, 2002, and published as Patent Application Publication US 2003/0221561 A1. The disclosures of all these related applications are incorporated herein by reference.

### FIELD OF THE INVENTION

The present invention relates generally to invasive medical devices and procedures, and specifically to devices and methods for controlling embolic flow in the bloodstream.

### BACKGROUND OF THE INVENTION

It is known in the art that acoustic waves traveling through a liquid exert a force on particles and bubbles suspended in liquid. The nature and strength of the interaction between acoustic waves and such particles is described, for example, by Yosioka and Kawasima, in “Acoustic Radiation Pressure on a Compressible Sphere,” Acustica 5 (1955), pages 167-173, which is incorporated herein by reference. This paper provides analytical formulas for calculating the acoustic force based on the parameters of the acoustic wave, the particles and the ambient liquid.

The above-mentioned Patent Application Publication US 2003/0221561 A1 describes ultrasonic devices that make use of acoustic radiation pressure in preventing emboli from reaching the brain during invasive cardiological procedures, such as cardiovascular surgery. (The term “embolus,” as used in the context of the present patent application and in the claims, refers to any abnormal particle circulating in the blood. Such particles may include, inter alia, cholesterol, platelet clumps, blood clots, calcium flecks, air bubbles, fat, and combinations of these components.) The published patent application describes various different devices for this purpose, including invasive devices that are designed for placement in the chest cavity during surgery and operate in combination with needle vents or other vent systems for removing diverted microbubbles.

In one embodiment described in US 2003/0221561 A1, a device for removing emboli from the bloodstream comprises a transducer associated with the exterior surface of the posterior side of the aorta in the general region of the transverse sinus. The transducer is powered to generate ultrasonic waves that are directed toward the anterior side of the aorta. A needle vent is inserted into the anterior side of the aorta downstream of the transverse sinus, so that emboli diverted by the transducer are removed through the needle vent.

### SUMMARY OF THE INVENTION

Embodiments of the present invention provide improved devices and methods for diversion of embolic flow within a blood vessel by transmitting ultrasonic waves into the vessel. These embodiments avoid the necessity of puncturing or otherwise invading the interior of the blood vessel, as is required in other methods that are known in the art.

The devices described hereinbelow are adapted particularly for deployment in the chest cavity, so as divert emboli flowing in the aortic arch into the descending aorta and away from the great origins of the neck vessels leading to the brain. Because the device is placed in close proximity to the target vessels, it can be aligned quickly and accurately by simple means. Such devices are useful particularly in preventing neurological damage that may occur due to release of emboli during cardiac surgery and other invasive cardiological procedures. The principles of the present invention may also be applied, however, for diversion of blood flow in other locations, such as the carotid bifurcations.

There is therefore provided, in accordance with an embodiment of the present invention, a device for controlling a flow of emboli in an aorta of a patient, the device including:

an ultrasonic transducer, which is configured to transmit an ultrasonic beam into the aorta in a vicinity of a great origin of a neck vessel; and

a driver circuit, which is coupled to drive the ultrasonic transducer to generate the ultrasonic beam at a frequency and power level sufficient to divert at least a target fraction of the emboli of a given type and size away from the neck vessel.

In a disclosed embodiment, the driver circuit is coupled to drive the ultrasonic transducer so as to reduce the flow of the emboli of the given size and type into the neck vessel by at least 80%, and the ultrasonic transducer is configured to transmit the ultrasonic beam so as to divert at least the target fraction of the emboli into the descending aorta.

In some embodiments, the device includes a holder, which is coupled to hold the ultrasonic transducer in proximity to the aorta. The holder may be fixed to a retractor, which is used to spread a sternum of the patient during open heart surgery. Typically, the holder is configured to hold the ultrasonic transducer on an anterior side of the aorta, so that the ultrasonic transducer transmits the ultrasonic beam in a posterior direction through the aorta.

In some embodiments, the ultrasonic beam is unfocused. In one embodiment, the ultrasonic beam has an intensity in the aorta of at least 0.3 W/cm², and the ultrasonic beam diverges from the transducer through the aorta.

Typically, the device includes a flexible coupler interposed between the transducer and the aorta. In some embodiments, the flexible coupler includes at least one of a gel and a polymer. In other embodiments, the flexible coupler includes a membrane, which contains a fluid for coupling the ultrasonic beam from the transducer to the aorta. In one of these embodiments, the device includes a housing, which contains the transducer and the fluid, wherein the membrane forms at least part of the housing, the housing including a fluid port for injecting the fluid into the housing while the transducer is fixed in proximity to the aorta. The device also includes a fluid circulation assembly coupled to the fluid port so as to cool the transducer by passage of the fluid through the housing, wherein the fluid circulation assembly includes a closed circuit.

In another embodiment, the device includes an acoustic waveguide, which is adapted to convey the ultrasonic beam from the ultrasonic transducer to the aorta. The acoustic waveguide has a distal end, which is configured to be brought into proximity with the aorta, and may include a diverging optic in a vicinity of the distal end.

In some embodiments, the driver circuit is adapted to actuate the ultrasonic transducer intermittently, responsively to variations in the flow of the emboli into the aorta. In one embodiment, the driver circuit is coupled to receive an indication of a heartbeat of the patient, and to actuate the ultrasonic transducer in synchronization with the heartbeat. In another embodiment, the driver circuit is adapted to actuate the ultrasonic transducer at a low power level during a first time period and at a high power level during a second time period, responsively to a variation in the flow of the emboli into the aorta associated with the second time period.

In further embodiments, the driver circuit is operative to actuate the ultrasonic transducer with pulsed excitation.

There is also provided, in accordance with an embodiment of the present invention, a device for controlling a flow of emboli in an aorta of a patient, the device including:

an ultrasonic transducer, which is configured to transmit an ultrasonic beam; and

a holder, including a proximal end that is adapted to be fixed to a retractor used to spread a sternum of the patient during open heart surgery, and a distal end that is coupled to hold the ultrasonic transducer in proximity to the aorta so that the transducer transmits the ultrasonic beam into the aorta during the surgery.

There is additionally provided, in accordance with an embodiment of the present invention, a device for conveying acoustical energy into tissue having an irregular shape, the device including:

an ultrasonic transducer, which is configured to transmit an ultrasonic beam; and

a flexible coupler interposed between the transducer and the tissue, the coupler including a matching material having acoustical properties similar to those of the tissue, which is adapted to deform to fit the irregular shape of the tissue so that the ultrasonic beam passes through the matching material into the tissue.

There is further provided, in accordance with an embodiment of the present invention, an ultrasonic assembly, including:

an ultrasonic transducer, which is configured to transmit an ultrasonic beam;

a housing, which contains the ultrasonic transducer and includes a coupler for coupling the ultrasonic beam into a target tissue;

cabling, having distal and proximal ends, the distal end coupled to the housing and including an electrical cable and fluid tubing; and

a cassette coupled to the proximal end of the cabling, the cassette including:
an electrical connector coupled to the electrical cable and adapted to be coupled to a power source for driving the transducer; and
a fluid reservoir coupled to the fluid tubing and containing a fluid for circulation through the housing via the tubing in order to cool the transducer.

In a disclosed embodiment, the assembly includes a console having a receptacle sized to receive the cassette, the console containing the power source for engaging the electrical connector and a mechanical drive for driving the circulation of the fluid. Typically, the console is adapted to drive the circulation of the fluid without contacting the fluid, which flows in a closed circuit through the tubing. Additionally or alternatively, the console may include a cooling device, which is positioned to thermally engage the fluid reservoir when the cassette is inserted in the receptacle. Further additionally or alternatively, the cassette includes an electronic device containing data regarding the assembly, and the console includes a wireless reader, which is coupled to read the data from the electronic device when the cassette is inserted in the receptacle. In one embodiment, the fluid reservoir and tubing are filled with the fluid and then hermetically sealed and sterilized before use of the assembly.

There is moreover provided, in accordance with an embodiment of the present invention, a method for controlling a flow of emboli in an aorta of a patient, the method including transmitting an ultrasonic beam into the aorta in a vicinity of a great origin of a neck vessel with an ultrasonic frequency and power level sufficient to divert at least a target fraction of the emboli of a given type and size away from the neck vessel.

In a disclosed embodiment, transmitting the ultrasonic beam includes actuating the ultrasonic beam intermittently, responsively to variations in the flow of the emboli into the aorta. Typically, actuating the ultrasonic beam includes receiving an indication of a heartbeat of the patient, and actuating the ultrasonic beam in synchronization with the heartbeat.

There is furthermore provided, in accordance with an embodiment of the present invention, a method for conveying acoustical energy into tissue having an irregular shape, the method including:

interposing a flexible coupler between an ultrasonic transducer and the tissue, the coupler including a matching material having acoustical properties similar to those of the tissue, which is adapted to deform to fit the irregular shape of the tissue; and

transmitting an ultrasonic beam from the ultrasonic transducer through the matching material into the tissue.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for diversion of emboli during a cardiac surgical procedure, in accordance with an embodiment of the present invention;

FIG. 2 is a schematic frontal view of the chest cavity of a patient during cardiac surgery, showing placement of an ultrasonic device for diversion of emboli, in accordance with an embodiment of the present invention;

FIG. 3 is a schematic side view of the chest cavity taken along a line III-III in FIG. 2, showing details of the placement of the ultrasonic device adjacent to the aorta, in accordance with an embodiment of the present invention;

FIG. 4 is a schematic, cross-sectional view taken along a line IV-IV in FIG. 3, illustrating acoustical coupling between the ultrasonic device and the aorta, in accordance with an embodiment of the present invention;

FIGS. 5A and 5B are schematic side and rear views of a cooled ultrasonic device for diversion of emboli, in accordance with an embodiment of the present invention;

FIG. 6A is a schematic side view of an assembly for ultrasonic diversion of emboli, in accordance with another embodiment of the present invention;

FIG. 6B is a schematic end view of the assembly of FIG. 6A, showing details of a connection between the assembly and a control console, in accordance with an embodiment of the present invention;

FIG. 7 is a schematic, pictorial illustration of an ultrasonic device for diversion of emboli during a cardiac surgical procedure, using a waveguide for transmission of acoustic energy, in accordance with an embodiment of the present invention; and

FIG. 8 is a schematic side view of an acoustic waveguide used in the device of FIG. 7, in accordance with an embodiment of the present invention.

### DETAILED DESCRIPTION OF EMBODIMENTS

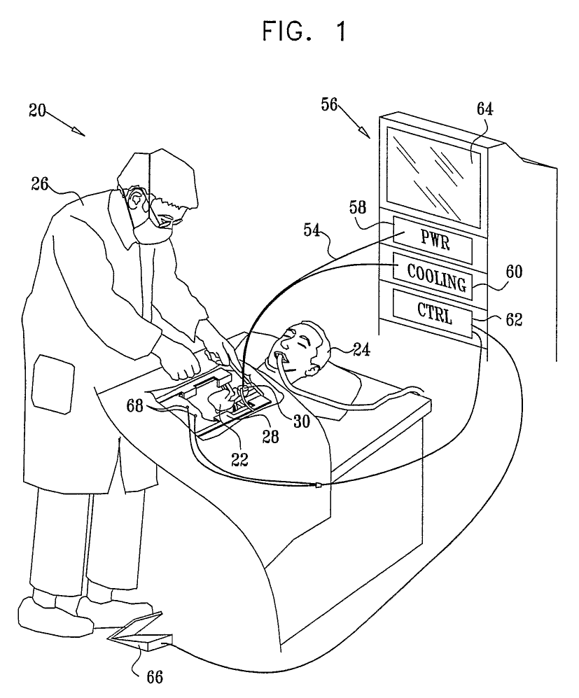

FIG. 1 is a schematic, pictorial illustration of a system 20 for diversion of emboli during an invasive procedure performed on a heart 22 of a patient 24, in accordance with an embodiment of the present invention. In this example, a surgeon 26 has opened the patient's chest by performing a median sternotomy, and has then attached a retractor 28 to spread the two parts of the sternum. The surgeon then cuts through the pericardium to expose the heart, as is known in the art. Before proceeding with the actual procedure on the heart, the surgeon places next to the aorta, in the most cranial part of the incision, an ultrasonic device 30 for diversion of emboli. Device 30 is deployed and operated to direct an ultrasonic beam into the aorta in such a way as to divert emboli in the aorta away from the great origins of the neck vessels. The structural and functional characteristics of device 30 are shown in detail in the figures that follow.

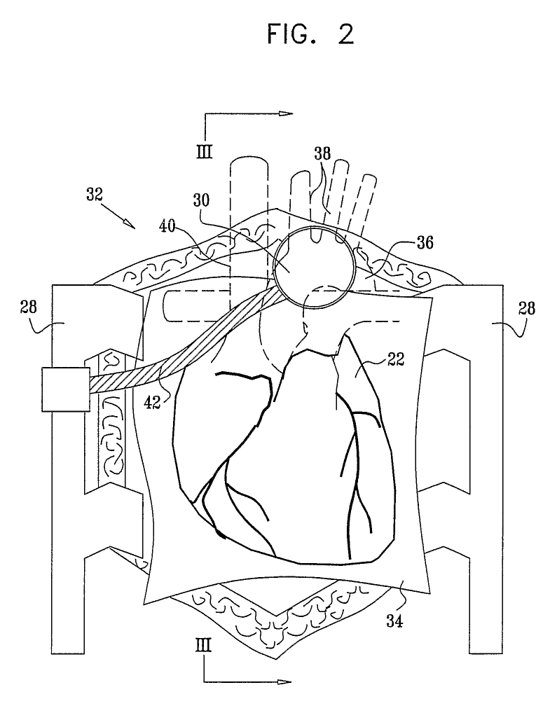

FIG. 2 is a schematic frontal view of a chest cavity 32 of patient 24, in accordance with an embodiment of the present invention. The clamps of retractor 28 hold the sternum open, and pericardium 34 is cut away to expose heart 22. Device 30 is placed against aorta 36, in proximity to the great origins of neck vessels 38, which include the innominate artery, the left common carotid artery and the left subclavian artery. (Superior vena cava 40 is shown for completeness.) In this embodiment, device 30 is held in place by an articulating arm 42, which is fastened to one of the clamps of retractor 28. Device 30 is thus held stably in the desired location and orientation in the upper chest cavity without interfering with the surgical field.

Additionally or alternatively, other means may be used to hold device 30 in place. For example, malleable wires attached to the device housing may be wrapped around the aorta and then sutured to prevent movement during the procedure.

FIG. 3 is a schematic side view of chest cavity 32 taken along a line III-III in FIG. 2. This figure illustrates further features of the mounting and operation of device 30, in accordance with an embodiment of the present invention. Note that device 30 is partly hidden beneath the patient's skin at the upper side of the open chest cavity (to the left in FIG. 3), although the entire device is revealed in FIG. 2 for the sake of visual clarity.

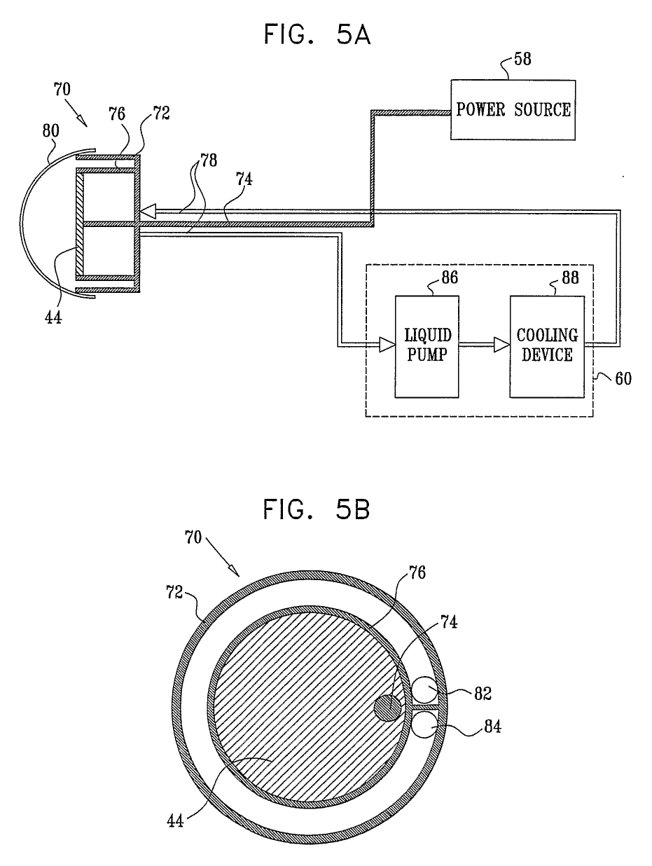

Device 30 comprises an ultrasonic transducer 44, such as a piezoelectric element or an array of such elements. Transducer 44 is coupled to aorta 36 through an acoustic coupler 46, in order to provide efficient energy transfer from the transducer to the blood vessel. Coupler 46 typically comprises a matching layer, i.e., a material that is acoustically transparent and possesses acoustical properties similar to those of soft tissue. For example, the material in coupler 46 may comprise an ultrasonic gel, silicone, polyethylene or even water (which may circulate to cool the transducer, as described below with reference to FIG. 5). As shown in FIG. 3, coupler 46 is sufficiently flexible to deform in order to fit the irregular shape of the tissue with which it is in contact. This deformation provides continuous coupling between device 30 and aorta 36, thus enhancing the efficiency of ultrasonic energy delivery.

In an alternative embodiment, not shown in the figures, the acoustic coupler of device 30 has a concave surface, which creates a closed cavity when the device is pressed against the target tissue. The cavity is then evacuated through a vacuum port in the device, causing the concave surface to flatten and adhere firmly to the tissue. The coupler is made flexible enough so that only a weak vacuum is necessary to achieve this effect. The vacuum is vented at the end of the procedure to permit the device to be removed.

FIG. 3 also shows the trajectory of a stream of emboli 48 emitted through aortic valve 50 (or possibly detached from the ascending aorta) into aorta 36. Actions of surgeon 26 during cardiac surgery, such as cannulation, de-cannulation and cross-clamping, are particularly likely to cause such emboli to be released into the bloodstream. In the absence of device 30, some of these emboli would simply be entrained in the branching blood flow into neck vessels 38. Device 30, however, is aimed so that the acoustic beam generated by transducer 44 exerts pressure on emboli 48 toward the descending aorta and away from the great origins of vessels 38. Thus, the emboli are diverted away from the neck vessels, and the brain of patient 24 is protected from neurological damage that could result if emboli 48 were to pass through one of vessels 38 and lodge in smaller blood vessels in the brain. Although the inventors have found the location and orientation shown in FIG. 3 to be optimal for diverting emboli into the descending aorta, other configurations can also be effective and are considered to be within the scope of the present invention. For example, ultrasonic transducers may be positioned at other locations and orientations along aorta 36 or in proximity to other blood vessels, in addition or alternatively to the location and orientation shown in FIG. 3.

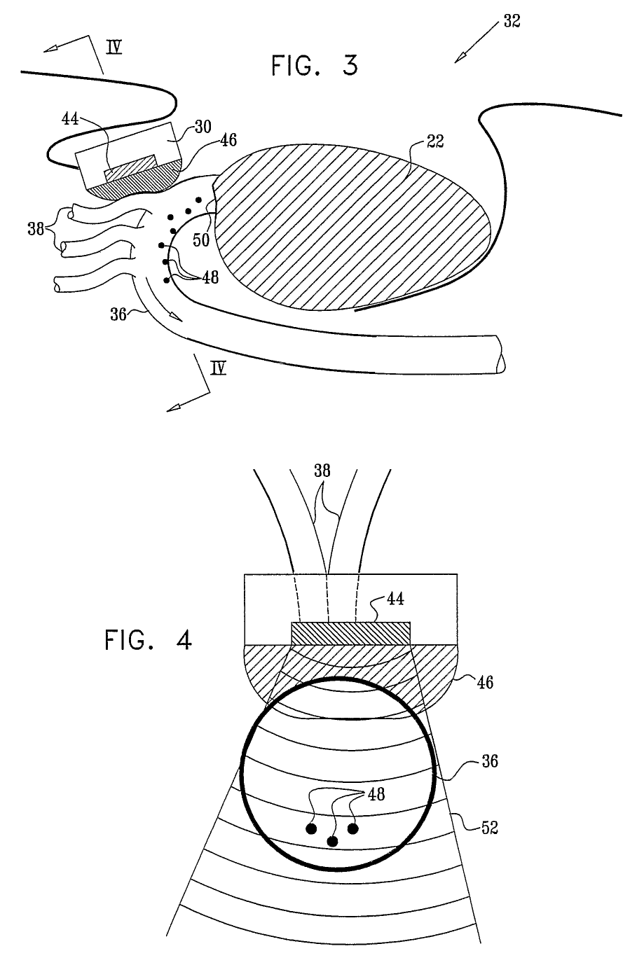

FIG. 4 is a schematic, cross-sectional view of device 30 and aorta 36, taken along a line IV-IV in FIG. 3. This figure shows a diverging acoustic beam 52 generated by transducer 44, in accordance with an embodiment of the present invention. The beam is directed toward the posterior part of the body (as illustrated in the preceding figures) and is wide enough to extend over at least the orifices of the first two branches of neck vessels 38, i.e., the innominate artery and the left common carotid artery. Typically, the width of beam 52 at this point is about 1 cm or more, and the average beam intensity is at least 0.3 W/cm² at a frequency of about 0.5 MHz or more.

The inventors found in bench and animal experiments in vivo that beam parameters of frequency 2.2 MHz and average intensity of 2 W/cm² were sufficient to divert at least 80% of a stream of polystyrene test particles 0.5 mm in diameter. In other words, under these beam conditions, the number of emboli of size 0.5 mm that enter the neck vessels is reduced by at least 80% relative to the number that would enter the neck vessels in the absence of device 30. A much lower intensity, as low as 0.5 W/cm² was sufficient to divert the vast majority of air bubbles.

Alternatively, other beam parameters may be used to divert a given target fraction of the particles of any other given size and type. In the context of the present patent application and in the claims, the “target fraction” refers to the percentage of the embolic particles that are to be diverted away from the neck vessels. The probability of neurological damage is reduced accordingly. The greater the beam intensity, the higher will be the percentage of emboli diverted. The higher the frequency, the smaller will be the minimum size of embolic particles that can be effectively diverted by the ultrasonic beam of device 30. For example, an ultrasonic beam with a frequency of 3 MHz is effective in diverting emboli whose size is 200 μm, while higher frequencies may be effective in diverting emboli as small as 100 μm. Higher frequencies, however, tend to have a stronger heating effect on the aorta and surrounding tissues. The optimal choice of ultrasound frequency and beam power will be apparent to those skilled in the art based on the criteria outlined herein. Ultrasound imaging of the blood vessels may be used to ascertain the effectiveness of a given frequency and beam power in diverting emboli of any given target size.

The use of diverging beam 52 is advantageous both in covering the entire cross-section of aorta 36 using a relatively small transducer, and in avoiding thermal damage to underlying tissues, such as the lungs and vertebrae. For example, assuming that the diameter of beam 52 at the vertebrae is twice the diameter in the aorta, the acoustic intensity at the vertebrae will then be only 25% of the intensity in the aorta. (The intensity generated at transducer 44, on the other hand, should be higher than the desired intensity in the aorta by a factor sufficient to compensate for the beam divergence.) To generate the diverging beam, transducer 44 may comprise a convex piezoelectric element or an array of piezoelectric elements mounted on a convex surface. Alternatively, the transducer may comprise a phased array of elements, which are driven electronically to generate the diverging beam. Any suitable diverging beam shape may be generated, using these or other transducer configurations known in the art.

In an alternative embodiment, not shown in the figures, transducer 44 generates a focused ultrasonic beam, which is aimed toward the great origins of neck vessels 38 in aorta 36 so as to deflect emboli 48 away from these specific locations. This approach is advantageous in reducing the total amount of ultrasonic energy to which the aorta is exposed, but it requires precise alignment of device 30. To aid in this alignment, device may comprise a Doppler ultrasound transducer, which detects the locations of the origins of the neck vessels based on the Doppler signature of the associated blood flow. The Doppler transducer may be mounted, for example, at the center of the power transducer that is used to generate the diverting beam. The power transducer is then aimed, either manually or automatically, so as to focus at the location indicated by the Doppler signal.

In still another embodiment, transducer 44 generates a non-focused ultrasound beam, whose diameter is roughly equal to or greater than the diameter of aorta 36. Such a beam may be generated, for example, by a piston-shaped transducer having a flat active element. In the context of the present patent application and in the claims, acoustic beams that are non-focused or substantially divergent within the aorta are referred to collectively as “unfocused beams.”

Returning now to FIG. 1, it can be seen that device 30 is connected by cabling 54 to a console 56. The console comprises a power driver circuit 58, which generates radio frequency (RF) energy for driving device 30, typically at the appropriate optimal frequency for transducer 44. Typically, the frequency generated by circuit 58 is in the range of 0.5 MHz or higher, with an electrical power output of at least 5 W for an unfocused beam. (The power level may be lower in embodiments that use a focused beam.) Alternatively, higher or lower frequencies and power levels may also be used, in accordance with therapeutic needs and technical constraints. As noted earlier, the frequency and power level are typically chosen by balancing the target particle size and the desired diversion percentage against the possible side effects of excessive tissue heating.

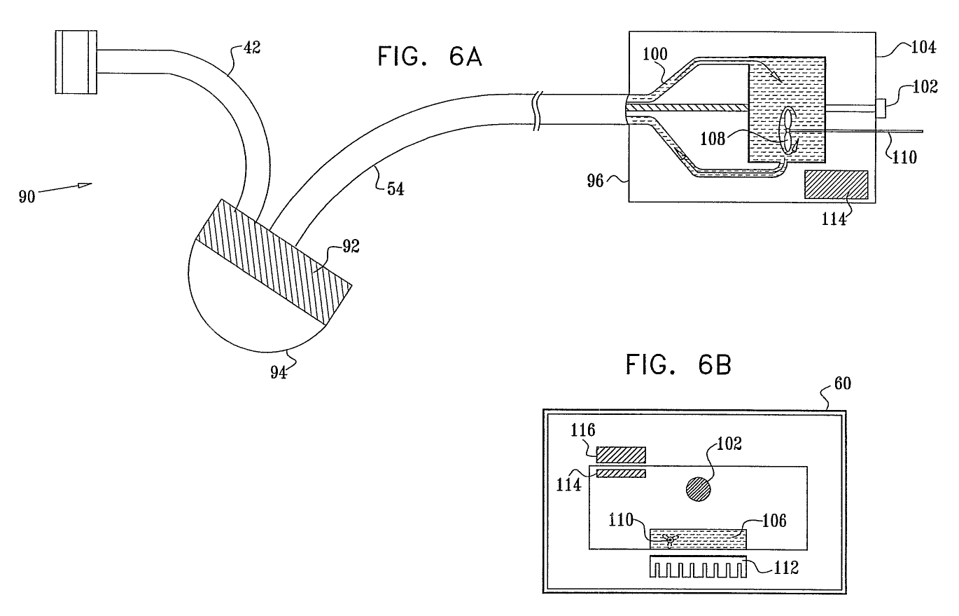

Cabling 54 may optionally comprise tubing for circulation of fluid between device 30 and a cooling unit 60. The purpose of the fluid circulation is to avoid overheating of transducer 44 during operation and to cool tissues with which acoustic coupler 46 is in contact. If the fluid circulates through coupler 46, the fluid can also serve as an effective coupling medium between the ultrasonic transducer and the tissue. These features of system 20 are described further hereinbelow with reference to FIGS. 5A, 5B, 6A and 6B.

The operation of system 20 is controlled by a control unit 62, which typically comprises a microprocessor with suitable interface and logic circuits for interacting with the other components of the system. Typically, the control unit activates and de-activates driver circuit 58 and cooling unit 60, based on parameters that are input to the system via a user interface 64. The user interface may comprise a touch screen, keyboard and/or pointing device (not shown). A remote control 66, such as a foot pedal, may also be provided to enable surgeon 26 (or another user) to switch device 30 on and off during surgery.

In order to reduce tissue heating, it is desirable that device 30 be controlled to emit an acoustic beam only when required, rather than operating continuously throughout the surgical procedure. In order to control device 30 in this manner, control unit 62 may be programmed to permit a number of different modes of operation, for example:
Continuous mode, in which operation of device 30 is controlled directly by surgeon 26 (or by another operator), typically using remote control 66. It is expected that the surgeon will actuate driver circuit 58 during surgical activities that are associated with high rates of embolism, such as cannulation, de-cannulation and cross-clamping.
Intermittent mode, for use particularly at acoustical power levels that are too high for continuous operation. In this case, the surgeon (or other operator) actuates driver circuit 58 just before beginning an activity that is likely to cause release of emboli. Control unit 62 permits the driver circuit to run for a predetermined length of time, typically between a few seconds and twenty minutes, depending on the acoustic beam frequency and power. At the end of the permitted time period, the control unit shuts the driver circuit off and prevents further operation of device 30 until a certain lockout period has elapsed.
Multi-power mode, for use in procedures in which air emboli are created throughout most of the duration of the procedure (emanating from a heart-lung machine, for example), and solid emboli are created in a short duration following aortic manipulations. For energy efficiency, the acoustic beam is active at low intensity for most or all of the procedure to divert the air bubbles. During aortic manipulations, the system is intermittently switched to high intensity for a short period of time (as in the intermittent mode above) to divert solid emboli.
Synchronized mode, for use in procedures (or parts of procedures) in which the patient's heart is beating. Control unit 62 may sense the heartbeat based on ECG signals from electrodes 68, for example, or other monitored physiological parameters. The control unit actuates device 30 to generate the acoustic beam in synchronization with the heartbeat so as to match the cardiac output function. Typically, the control unit turns on the beam at full power only during peak systolic flow, while the beam power is reduced (or even turned off) during the remainder of the heart cycle, during which the rate of blood flow through aortic valve 50 is much lower. This mode of operation reduces the average acoustic power applied to aorta 36 by a factor of 3-4 relative to the continuous mode.

In all of the above modes, when device 30 is actuated, it may be driven by either continuous wave (CW) or pulsed excitation, i.e., with a duty cycle less than 100%. When pulsed excitation is used, the radiation pressure exerted on the emboli is pulsed. The emboli can thus accumulate diversion by virtue of momentum acquired during previous pulses, resulting in more efficient diversion at lower average acoustic power as compared with continuous excitation. Another advantage of pulsed excitation is that it broadens the spectral band of the emitted acoustic wave, resulting in a more homogeneous beam in the near field zone.

As noted above, cooling unit 60 is optional, and the need for such a unit depends on the configuration of device 30 and on the efficiency and mode of operation of transducer 44. Referring, for example, to the configuration shown in FIG. 4, let us assume that transducer 44 generates 40 W of acoustic power with an efficiency of 80%, meaning that the transducer generates 10 W of heat. Assuming coupler 46 to comprise a gel pad of volume 40 cm³, the heat generated by transducer 44 will cause the temperature of the gel pad to increase by about 3.5° C. per minute of operation. Thus, as long as actuation of device 30 is limited to periods of no more than a few minutes, separated by inactive periods of at least equal length to permit the gel pad to cool, device 30 may operate without external cooling. When high enough acoustic power is applied so that passive temperature dissipation is insufficient, or transducer 44 is less efficient, an external cooling circuit may be used, such as those described below.

FIGS. 5A and 5B schematically illustrate a fluid-cooled ultrasonic device 70 for diversion of emboli, in accordance with an embodiment of the present invention. FIG. 5A shows a side view of device 70, together with elements of console 56, while FIG. 5B is a rear view of the device. Device 70 may be used in system 20 in substantially the same manner as device 30, and has similar properties to device 30 with the exception of the specific points described hereinbelow. In device 70, transducer 44 is contained inside a housing 72, which is filled with a circulating fluid supplied by cooling unit 60. The transducer receives RF power from circuit 58 via a power feed-through 74 in a mount 76, which fixes the transducer to housing 72. The housing typically comprises a rigid biocompatible plastic, such as an acrylic, polycarbonate or fluorocarbon material, polyetheretherketone (PEEK) or a biocompatible metal, such as stainless steel, titanium or aluminum. The front of the housing comprises an acoustic window 80, through which acoustic waves from transducer 44 are emitted. The window typically comprises a thin, flexible, acoustically-transparent membrane, such as latex, silicone, polyurethane or polyethylene.

Cooling unit 60 pumps fluid through housing 72 via tubing 78, which is connected to an inlet port 82 and an outlet port 84 of the housing. The fluid flows through the space between housing 72 and mount 76 into and out of the region between transducer 44 and window 80. (The area inside mount 76 may be filled with air.) The fluid in this case performs the role of coupler 46 in the preceding embodiment. In other words, the fluid both cools transducer 44 and serves as the flexible matching layer between the transducer and the target tissues in the body of patient 24. The housing is hermetically sealed except for ports 82 and 84.

Typically, window 80 is slack until housing 72 is pressurized with the fluid, which then presses the window against the adjacent tissues so that the fluid matching layer inside the housing conforms to the target tissues. Outlet port 84 may be narrower than inlet port 82 in order to facilitate pressurization of the housing. In an alternative embodiment, not shown in the figures, the sides of the transducer housing also comprise thin, flexible material, like window 80, so that the housing inflates like a balloon when pressurized with fluid. Other materials and methods of construction will be apparent to those skilled in the art.

Cooling unit 60 comprises a pump 86, which circulates the fluid between housing 72 and a cooling device 88, such as a refrigerator or heat exchanger. The cooling unit thus ensures both that device 70 is kept at the proper temperature and that housing 72 is pressurized in order to inflate window 80. Rapid flow of fluid through housing 72 also removes air bubbles that otherwise might disperse some of the acoustic energy emitted by transducer 44. While the combined acoustic matching and cooling functions performed by the fluid in housing 72 are particularly useful when device 70 is used for diversion of emboli in the aorta, this sort of transducer assembly and housing can also be used in other medical ultrasound applications, particularly applications involving high-power acoustic sonication.

Other schemes may also be used for cooling transducer 44. For example, cooled liquid or gas (or both) may flow through the transducer housing on the back side of the transducer, while the front side is coupled to the target tissue through a gel or polymer matching layer. As another example, the back side of the transducer may be air-cooled, while cooling fluid flows over the front of the transducer. Other cooling schemes will be apparent to those skilled in the art.

FIG. 6A is a schematic side view of a disposable transducer assembly 90, in accordance with another embodiment of the present invention. Assembly 90 comprises an ultrasonic device 92, which contains a transducer (as shown in the preceding figures) and an acoustic coupler 94, along with arm 42, as described above. The acoustic coupler may comprise any suitable material, such as polymer, gel or liquid, either stationary or flowing, as described above. Device 92 is connected by cabling 54 to a cassette 96, which is designed to be inserted into and mate with a receptacle in cooling unit 60. Assembly 90 is provided as an integral, sealed, sterile unit, intended to be used once and then disposed of thereafter.

Cabling 54 comprises electrical cable 98, for providing power to the transducer in device 92, and fluid hoses 100, through which liquid or gas circulates to and from device 92 in order to cool the transducer. Cable 98 terminates in a connector 102 at a proximal side 104 of cassette 96. The fluid in hoses 100 is pumped through a cooling reservoir 106 in cassette 96 by a rotor 108. The rotor is driven through a shaft 110, which likewise terminates at the proximal side of the cassette. Alternatively, a section of hose 100 may protrude at one of the sides of the cassette to engage a roller pump in cooling unit 60. In either case, the fluid in assembly flows in a closed circuit. Cassette 96 may thus be hermetically sealed (with suitable feedthroughs for cabling 54, connector 102 and shaft 110), so that the fluid inside assembly 90 never comes into contact with cooling unit 60, and the sterility of device 92 is maintained.

FIG. 6B is a schematic end view of cassette 96 inside cooling unit 60, seen from proximal side 104 of the cassette. Connector 102 and shaft 110 mate with suitable electrical and mechanical drive connectors (not shown) inside the cooling unit when the cassette is plugged into the mating receptacle. Although cassette 96 is shown in this figure to be rectangular in shape, other shapes of the cassette and the mating receptacle, such as a cylindrical shape, are also possible. Reservoir 106 is positioned inside cassette 96 next to one of the side walls of the cassette, which comes in contact with a cooling device 112, such as a Peltier cooler, in unit 60. The fluid in the reservoir is thus cooled by transfer of heat through the side wall of the cassette to the cooling device. Optionally, cassette 96 comprises an electronic identification chip 114, containing information that can be read out by a wireless reader 116 in cooling unit 60 in order to verify that assembly 90 is of the proper type and is used no more than once.

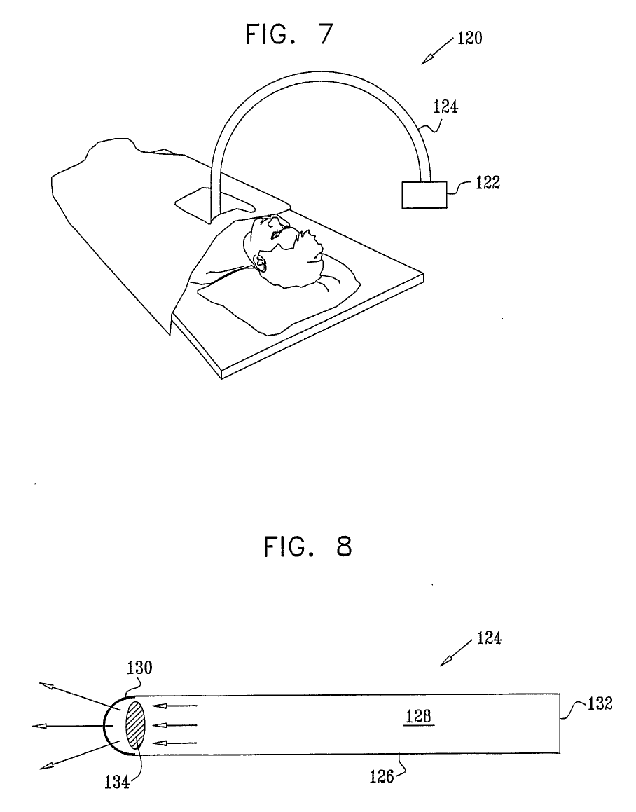

FIG. 7 is a schematic, pictorial illustration showing an ultrasonic device 120 for diversion of emboli during a cardiac surgical procedure, in accordance with yet another embodiment of the present invention. In this embodiment, a transducer 122 is remotely located, away from the surgical site. Ultrasonic waves are transferred from the transducer to the surgical site via an acoustic waveguide 124. This approach alleviates the need to sterilize the ultrasonic transducer, and also reduces mechanical and thermal problems and constraints associated with positioning the transducer in the chest cavity.

FIG. 8 is a schematic side view of waveguide 124, in accordance with an embodiment of the present invention. The waveguide comprises a hollow shell 126, made of a flexible, non-kinking material such as a thin plastic or metal. The shell is filled with a coupling material 128, such as a liquid, gel or polymer, having low acoustic attenuation and acoustical properties similar to the target tissue of patient 24. For example, material 128 may comprise degassed water or acoustic gel. Material 128 may be static or, if the material is liquid, it may be circulated through shell 126 by a suitable pump and cooling system (not shown).

Shell 126 should be substantially thinner than the acoustic wavelength of the ultrasonic waves generated by transducer 122 in order to avoid transfer of acoustical energy from material 128 to the shell. If material 128 comprises a liquid or gel, the distal and proximal ends of waveguide 124 are also closed by respective membranes 130 and 132. Transducer 122 is coupled to the waveguide through membrane 132, while membrane 130 contacts the target tissue in the patient's body and deforms to couple with the target tissue.

Optionally, waveguide 124 comprises optics, such as a diverging lens 134, for generating a diverging output beam, as shown, for example, in FIG. 4. The shape and refractive index of lens 134 are chosen so as to engender the desired divergence angle in the ultrasonic beam. The material in lens 134 is chosen to have acoustic impedance close to the impedance of material 128 in order to minimize back-reflection from the lens. Alternatively, a divergent beam may be created at the output of the waveguide by forming the output side of the waveguide in a trumpet-like shape (not shown).

Although the ultrasonic devices described hereinabove are designed specifically for use in diversion of emboli in the aorta, the principles of these devices may be applied, mutates mutandis, for diversion of emboli in other locations, such as the carotid bifurcation, as well as in other invasive and non-invasive applications of medical ultrasound. Similarly, although certain specific device designs are shown and described hereinabove, the therapeutic principles embodied in these devices may also be implemented using other device designs, as will be apparent to those skilled in the art.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A device for controlling a flow of emboli in an aorta of a patient, the device comprising:
an ultrasonic transducer, which is configured to transmit an ultrasonic beam into the aorta in a vicinity of a great origin of a neck vessel; and
a driver circuit, which is coupled to drive the ultrasonic transducer to generate the ultrasonic beam at a frequency and power level sufficient to divert at least a target fraction of the emboli of a given type and size away from the neck vessel.

2. The device according to claim 1, wherein the driver circuit is coupled to drive the ultrasonic transducer so as to reduce the flow of the emboli of the given size and type into the neck vessel by at least 80%.

3. The device according to claim 1, wherein the ultrasonic transducer is configured to transmit the ultrasonic beam so as to divert at least the target fraction of the emboli into the descending aorta.

4. The device according to claim 1, and comprising a holder, which is coupled to hold the ultrasonic transducer in proximity to the aorta.

5. The device according to claim 4, wherein the holder is adapted to be fixed to a retractor, which is used to spread a sternum of the patient during open heart surgery.

6. The device according to claim 4, wherein the holder is configured to hold the ultrasonic transducer on an anterior side of the aorta, so that the ultrasonic transducer transmits the ultrasonic beam in a posterior direction through the aorta.

7. The device according to claim 1, wherein the ultrasonic beam is unfocused.

8. The device according to claim 7, wherein the ultrasonic beam has an intensity in the aorta of at least 0.3 W/cm².

9. The device according to claim 7, wherein the ultrasonic beam diverges from the transducer through the aorta.

10. The device according to claim 1, and comprising a flexible coupler interposed between the transducer and the aorta.

11. The device according to claim 10, wherein the flexible coupler comprises at least one of a gel and a polymer.

12. The device according to claim 10, wherein the flexible coupler comprises a membrane, which contains a fluid for coupling the ultrasonic beam from the transducer to the aorta.

13. The device according to claim 12, and comprising a housing, which contains the transducer and the fluid, wherein the membrane forms at least part of the housing, the housing comprising a fluid port for injecting the fluid into the housing while the transducer is fixed in proximity to the aorta.

14. The device according to claim 13, and comprising a fluid circulation assembly coupled to the fluid port so as to cool the transducer by passage of the fluid through the housing.

15. The device according to claim 14, wherein the fluid circulation assembly comprises a closed circuit.

16. The device according to claim 1, and comprising an acoustic waveguide, which is adapted to convey the ultrasonic beam from the ultrasonic transducer to the aorta.

17. The device according to claim 16, wherein the acoustic waveguide has a distal end, which is configured to be brought into proximity with the aorta, and comprises a diverging optic in a vicinity of the distal end.

18. The device according to claim 1, wherein the driver circuit is adapted to actuate the ultrasonic transducer intermittently, responsively to variations in the flow of the emboli into the aorta.

19. The device according to claim 18, wherein the driver circuit is coupled to receive an indication of a heartbeat of the patient, and to actuate the ultrasonic transducer in synchronization with the heartbeat.

20. The device according to claim 1, wherein the driver circuit is adapted to actuate the ultrasonic transducer at a low power level during a first time period and at a high power level during a second time period, responsively to a variation in the flow of the emboli into the aorta associated with the second time period.

21. The device according to claim 1, wherein the driver circuit is operative to actuate the ultrasonic transducer with pulsed excitation.

22. A device for controlling a flow of emboli in an aorta of a patient, the device comprising:
an ultrasonic transducer, which is configured to transmit an ultrasonic beam; and
a holder, comprising a proximal end that is adapted to be fixed to a retractor used to spread a sternum of the patient during open heart surgery, and a distal end that is coupled to hold the ultrasonic transducer in proximity to the aorta so that the transducer transmits the ultrasonic beam into the aorta during the surgery.

23. An ultrasonic assembly, comprising:
an ultrasonic transducer, which is configured to transmit an ultrasonic beam;
housing, which contains the ultrasonic transducer and comprises a coupler for coupling the ultrasonic beam into a target tissue;
cabling, having distal and proximal ends, the distal end coupled to the housing and comprising an electrical cable and fluid tubing; and
a cassette coupled to the proximal end of the cabling, the cassette comprising:
an electrical connector coupled to the electrical cable and adapted to be coupled to a power source for driving the transducer; and
a fluid reservoir coupled to the fluid tubing and containing a fluid for circulation through the housing via the tubing in order to cool the transducer.

24. The assembly according to claim 23, and comprising a console having a receptacle sized to receive the cassette, the console containing the power source for engaging the electrical connector and a mechanical drive for driving the circulation of the fluid.

25. The assembly according to claim 24, wherein the console is adapted to drive the circulation of the fluid without contacting the fluid, which flows in a closed circuit through the tubing.

26. The assembly according to claim 24, wherein the console comprises a cooling device, which is positioned to thermally engage the fluid reservoir when the cassette is inserted in the receptacle.

27. The assembly according to claim 24, wherein the cassette comprises an electronic device containing data regarding the assembly, and wherein the console comprises a wireless reader, which is coupled to read the data from the electronic device when the cassette is inserted in the receptacle.

28. The assembly according to claim 23, wherein the fluid reservoir and tubing are filled with the fluid and then hermetically sealed and sterilized before use of the assembly.

29. A method for controlling a flow of emboli in an aorta of a patient, the method comprising transmitting an ultrasonic beam into the aorta in a vicinity of a great origin of a neck vessel with an ultrasonic frequency and power level sufficient to divert at least a target fraction of the emboli of a given type and size away from the neck vessel.

30. The method according to claim 29, wherein the ultrasonic frequency and power level are sufficient to reduce the flow of the emboli of the given size and type into the neck vessel by at least 80%.

31. The method according to claim 29, wherein transmitting the ultrasonic beam comprises diverting at least the target fraction of the emboli into the descending aorta.

32. The method according to claim 29, wherein transmitting the ultrasonic beam comprises positioning an ultrasonic transducer on an anterior side of the aorta, and transmitting the ultrasonic beam from the ultrasonic transducer in a posterior direction through the aorta.

33. The method according to claim 29, wherein transmitting the ultrasonic beam comprises transmitting an unfocused beam.

34. The method according to claim 33, wherein the ultrasonic beam has an intensity in the aorta of at least 0.3 W/cm².

35. The method according to claim 33, wherein transmitting the unfocused beam comprises transmitting a diverging beam.

36. The method according to claim 29, wherein transmitting the ultrasonic beam comprises positioning an ultrasonic transducer to transmit the beam, and interposing a flexible coupler between the transducer and the aorta so as to couple the beam into the aorta.

37. The method according to claim 36, wherein the flexible coupler comprises at least one of a gel and a polymer.

38. The method according to claim 36, wherein the flexible coupler comprises a membrane, which contains a fluid for coupling the ultrasonic beam from the transducer to the aorta.

39. The method according to claim 38, and comprising circulating the fluid through a housing of the transducer so as to cool the transducer.

40. The method according to claim 29, wherein transmitting the ultrasonic beam comprises conveying the ultrasonic beam from an ultrasonic transducer through an ultrasonic waveguide to the aorta.

41. The method according to claim 29, wherein transmitting the ultrasonic beam comprises actuating the ultrasonic beam intermittently, responsively to variations in the flow of the emboli into the aorta.

42. The method according to claim 41, wherein actuating the ultrasonic beam comprises receiving an indication of a heartbeat of the patient, and actuating the ultrasonic beam in synchronization with the heartbeat.

43. The method according to claim 29, wherein transmitting the ultrasonic beam comprises actuating the ultrasonic transducer at a low power level during a first time period and at a high power level during a second time period, responsively to a variation in the flow of the emboli into the aorta associated with the second time period.

44. The method according to claim 29, wherein transmitting the ultrasonic beam comprises actuating the ultrasonic beam with pulsed excitation.
